(19) United States
(12) Patent Application Publication
(10) Pub. No.: US 2015/0119541 A1
(43) Pub. Date: Apr. 30, 2015

(54) PROCESS FOR THE PRODUCTION OF METHACRYLIC ACID AND ITS DERIVATIVES AND POLYMERS PRODUCED THEREFROM

(71) Applicant: Lucite International UK Limited, Southampton, Hampshire (GB)

(72) Inventors: Graham Ronald Eastham, Durham (GB); David William Johnson, Durham (GB); Mark Waugh, Durham (GB)

(21) Appl. No.: 14/396,365

(22) PCT Filed: Apr. 26, 2013
(86) PCT No.: PCT/GB2013/051080

§ 371 Date: Oct. 22, 2014

(30) Foreign Application Priority Data
Apr. 27, 2012 (GB) 1207388.8

(51) Int. Cl. C07C 51/38; C08F 20/28; C08F 20/32; C08F 20/34; C08F 20/06; C08F 20/18

(52) U.S. Cl. CPC C07C 51/38; C08F 20/06; C08F 20/18; C08F 20/32; C08F 20/34; C08F 20/28
USPC 526/273; 562/598; 526/317.1; 526/329.7; 526/282; 526/320; 526/318.2; 526/307.6; 526/307.7; 526/318.4; 526/328.5; 526/329.2; 526/329.1; 526/325; 528/75; 526/312; 526/318.6; 526/318.42; 526/329.3; 526/329.5

(57) ABSTRACT

A process for the production of methacrylic acid or esters thereof by the base catalysed decarboxylation of at least one dicarboxylic acid selected from itaconic, citraconic or mesaconic acid or mixtures thereof in an aqueous reaction medium is described. The decarboxylation is carried out at a temperature in the range from 200° C. and up to 239° C. The methacrylic acid is isolated from the aqueous reaction medium by a purification process which does not include introducing an organic solvent to the aqueous reaction medium for solvent extraction of the methacrylic acid into an organic phase. A method of preparing polymers or copolymers of methacrylic acid or methacrylic acid esters is also described.

The present invention relates to a process for the production of methacrylic acid or derivatives such as esters thereof by the decarboxylation of itaconic acid or a source thereof in the presence of base catalysts, in particular, but not exclusively, a process for the production of methacrylic acid or methyl methacrylate.

Methacrylic acid (MAA) and its methyl ester, methyl methacrylate (MMA) are important monomers in the chemical industry. Their main application is in the production of plastics for various applications. The most significant polymerisation application is the casting, moulding or extrusion of polymethyl methacrylate (PMMA) to produce high optical clarity plastics. In addition, many copolymers are used; important copolymers are copolymers of methyl methacrylate with α-methyl styrene, ethyl acrylate and butyl acrylate. Currently MMA (and MAA) is produced entirely from petrochemical feedstocks.

Conventionally, MMA has been produced industrially via the so-called acetone-cyanohydrin route. The process is capital intensive and produces MMA from acetone and hydrogen cyanide at a relatively high cost. The process is effected by forming acetone cyanohydrin from the acetone and hydrogen cyanide: dehydration of this intermediate yields methacrylamide sulphate, which is then hydrolysed to produce MAA. The intermediate cyanohydrin is converted with sulphuric acid to a sulphate ester of the methacrylamide, methanolysis of which gives ammonium bisulphate and MMA. However, this method is not only expensive, but both sulphuric acid and hydrogen cyanide require careful and expensive handling to maintain a safe operation and the process produces large amounts of ammonium sulphate as a by-product. Conversion of this ammonium sulphate either to a useable fertilizer or back to sulphuric acid requires high capital cost equipment and significant energy costs.

Alternatively, in a further process, it is known to start with an isobutylene or, equivalently, t-butanol reactant which is then oxidized to methacrolein and then to MAA.

An improved process that gives a high yield and selectivity and far fewer by-products is a two stage process known as the Alpha process. Stage I is described in WO96/19434 and relates to the use of 1,2-bis-(di-t-butylphosphinomethyl)benzene ligand in the palladium catalysed carbonylation of ethylene to methyl propionate in high yield and selectivity. The applicant has also developed a process for the catalytic conversion of methyl propionate (MEP) to MMA using formaldehyde. A suitable catalyst for this is a caesium catalyst on a support, for instance, silica. This two stage process although significantly advantageous over the competitive processes available still nevertheless relies on ethylene feed stocks predominantly from crude oil and natural gas, albeit bioethanol is also available as a source of ethylene.

For many years, biomass has been offered as an alternative to fossil fuels both as a potential alternative energy resource and as an alternative resource for chemical process feedstocks. Accordingly, one obvious solution to the reliance on fossil fuels is to carry out any of the known processes for the production of MMA or MAA using a biomass derived feedstock.

In this regard, it is well known that syngas (carbon monoxide and hydrogen) can be derived from Biomass and that methanol can be made from syngas. Several Industrial plants produce methanol from syngas on this basis, for example, at Lausitzer Analytik GmbH Laboratorium für Umwelt and Brennstoffe Schwarze Pumpe in Germany and Biomethanol Chemie Holdings, Delfzijl, Netherlands. Nouri and Tillman, Evaluating synthesis gas based biomass to plastics (BTP) technologies, (ESA-Report 2005:8 ISSN 1404-8167) teach the viability of using methanol produced from synthesis gas as a direct feedstock or for the production of other feedstocks such as formaldehyde. There are also many patent and non-patent publications on production of syngas suitable for production of chemicals from biomass.

The production of ethylene by dehydration of biomass derived ethanol is also well established with manufacturing plants in, especially, Brazil.

The production of propionic acid from carbonylation of ethanol and the conversion of biomass derived glycerol to molecules such as acrolein and acrylic acid is also well established in the patent literature.

Thus ethylene, carbon monoxide and methanol have well established manufacturing routes from biomass. The chemicals produced by this process are either sold to the same specification as oil/gas derived materials, or are used in processes where the same purity is required.

Thus in principle there is no barrier to operation of the so called Alpha process above to produce methyl propionate from Biomass derived feedstocks. In fact, its use of simple feedstocks such as ethylene, carbon monoxide and methanol rather sets it apart as an ideal candidate.

In this regard, WO2010/058119 relates explicitly to the use of biomass feedstocks for the above Alpha process and the catalytic conversion of methyl propionate (MEP) produced to MMA using formaldehyde. These MEP and formaldehyde feedstocks could come from a biomass source as mentioned above. However, such a solution still involves considerable processing and purification of the biomass resource to obtain the feedstock which processing steps themselves involve the considerable use of fossil fuels.

Further, the Alpha process requires multiple feedstocks in one location which can lead to availability issues. It would therefore be advantageous if any biochemical route avoided multiple feedstocks or lowered the number of feedstocks.

Therefore, an improved alternative non-fossil fuel based route to acrylate monomers such as MMA and MAA is still required.

PCT/GB2010/052176 discloses a process for the manufacture of aqueous solutions of acrylates and methacrylates respectively from solutions of malate and citramalate acids and their salts.

Carlsson et al., Ind. Eng. Chem. Res. 1994, 33, 1989-1996 has disclosed itaconic acid decarboxylation to MAA at high temperatures of 360° C. and with a maximum yield of 70%. Carlsson found a decrease in selectivity in moving from 360 to 350° C. under ideal conditions.

Generally, for industrial processes a high selectivity is required to avoid generation of unwanted by-products which would eventually render a continuous process untenable. For this purpose, particularly for a continuous process, selectivity for the desired product should exceed 90%.

Surprisingly, it has now been discovered that high selectivity to MAA formation in excess of 90% in the decarboxylation of itaconic acid and other itaconic equilibrated acids can be achieved at significantly lower temperatures.

According to a first aspect of the present invention there is provided a process for the production of methacrylic acid or esters thereof by the base catalysed decarboxylation of at least one dicarboxylic acid selected from itaconic, citraconic or mesaconic acid or mixtures thereof in an aqueous reaction medium, wherein the decarboxylation is carried out at a temperature in the range from 200° C. and up to 239° C. and wherein the methacrylic acid is isolated from the aqueous reaction medium by a purification process which does not include introducing an organic solvent to the said aqueous reaction medium for solvent extraction of the methacrylic acid into an organic phase.

Preferably, the base catalysed decarboxylation of the at least one dicarboxylic acid takes place in the temperature ranges between 205 and up to 235° C., more preferably, between 210 and 230° C.

The term introducing an organic solvent to the said aqueous reaction medium includes contacting an organic solvent with the aqueous reaction medium.

Suitable processes to isolate the methacrylic acid from the aqueous reaction medium may be selected from distillation, fractional crystallisation (this can include crystallisation of the free acid or crystallisation of a salt of the acid such as the group I and II metal salt, for example the calcium salt followed by acidification to regenerate the free MAA). Crystallisation may be preceded by suitable separation such as ion exchange chromatography, for example, adsorption of MAA on a basic anion exchanger such as an amine ion exchange resin followed by desorption with strong acid, for example HCl. A further suitable technique is bipolar membrane electrodialysis (BPMED) to increase the purity of the MAA prior to crystallisation, for example by forming MAA and NaOH from sodium methacrylate. A still further isolation technique involves esterification to the alkyl ester such as the methyl, ethyl or butyl ester to give MMA, EMA or BMA followed by distillation and optional subsequent hydrolysis to regenerate the MAA.

The dicarboxylic acid(s) reactants and the base catalyst need not necessarily be the only compounds present. The dicarboxylic acid(s) together with any other compounds present are generally dissolved in an aqueous solution for the base catalysed thermal decarboxylation.

Advantageously, carrying out the decarboxylation at lower temperatures prevents the production of significant amounts of by-products which may be difficult to remove and may cause further purification and processing problems in an industrial process. Therefore, the process provides a surprisingly improved selectivity in this temperature range. Furthermore, lower temperature decarboxylation uses less energy and thereby creates a smaller carbon footprint than high temperature decarboxylations.

The dicarboxylic acids are available from non-fossil fuel sources. For instance, the itaconic, citraconic or mesaconic acids could be produced from a source of pre-acids such as citric acid or isocitric acid by dehydration and decarboxylation at suitably high temperatures or from aconitic acid by decarboxylation at suitably high temperatures. It will be appreciated that a base catalyst is already present so that the source of pre-acid dehydration and/or decomposition may potentially be base catalysed under such suitable conditions. Citric acid and isocitric acid may themselves be produced from known fermentation processes and aconitic acid may be produced from the former acids. Accordingly, the process of the invention may provide a biological or substantially biological route to generate methacrylates directly whilst minimising reliance on fossil fuels.

As detailed above, the base catalysed decarboxylation of the at least one dicarboxylic acid takes place at less than 240° C., more typically, at less than 235° C., more preferably, at up to 235° C., most preferably at up to 230° C. In any case, a preferred lower temperature for the process of the present invention is 205° C., more preferably, 210° C., most preferably, 215° C. Preferred temperature ranges for the process of the present invention are between 205° C. and up to 235° C., more preferably, between 210° C. and 235° C.

Preferably, the reaction takes place at a temperature at which the reaction medium is in the liquid phase. Typically, the reaction medium is an aqueous solution.

Preferably, the base catalysed decarboxylation takes place with the dicarboxylic acid reactants and preferably the base catalyst in aqueous solution.

To maintain the reactants in the liquid phase under all above temperature conditions the decarboxylation reaction of the at least one dicarboxylic acid is carried out at suitable pressures in excess of atmospheric pressure. Suitable pressures which will maintain the reactants in the liquid phase in the above temperature ranges are greater than 225 psia, more suitably, greater than 240 psia, most suitably, greater than 260 psia and in any case at a higher pressure than that below which the reactant medium will boil. There is no upper limit of pressure but the skilled person will operate within practical limits and within apparatus tolerances, for instance, at less than 10,000 psia, more typically, at less than 5,000 psia, most typically, at less than 4000 psia.

Preferably, the above reaction is at a pressure of between about 225 and 10000 psia. More preferably, the reaction is at a pressure of between about 240 and 5000 psia and yet more preferably between about 260 and 3000 psia.

In a preferred embodiment, the above reaction is at a pressure at which the reaction medium is in the liquid phase.

Preferably, the reaction is at a temperature and pressure at which the reaction medium is in the liquid phase.

As mentioned above, the catalyst is a base catalyst.

Preferably, the catalyst comprises a source of OH⁻ ions. Preferably, the base catalyst is selected from the group consisting of a metal oxide, hydroxide, carbonate, acetate (ethanoate), alkoxide, hydrogencarbonate; or salt of a decomposable di- or tri-carboxylic acid; or a quaternary ammonium compound of one of the above; or one or more amines; more preferably a Group I or Group II metal oxide, hydroxide, carbonate, acetate, alkoxide, hydrogencarbonate or salt of a di- or tri-carboxylic acid or methacrylic acid.

Preferably, the base catalyst is selected from one or more of the following: LiOH, NaOH, KOH, Mg(OH)₂, Ca(OH)₂, Ba(OH)₂, CsOH, Sr(OH)₂, RbOH, NH₄OH, Li₂CO₃, Na₂CO₃, K₂CO₃, Rb₂CO₃, Cs₂CO₃, MgCO₃, CaCO₃, SrCO₃, BaCO₃, (NH₄)₂CO₃, LiHCO₃, NaHCO₃, KHCO₃, RbHCO₃, CsHCO₃, Mg(HCO₃)₂, Ca(HCO₃)₂, Sr(HCO₃)₂, Ba(HCO₃)₂, NH₄HCO₃, Li₂O, Na₂O, K₂O, Rb₂O, Cs₂O, MgO, CaO, SrO, BaO, Li(OR¹), Na(OR¹), K(OR¹), Rb(OR¹), Cs(OR¹), Mg(OR¹)₂, Ca(OR¹)₂, Sr(OR¹)₂, Ba(OR¹)₂, NH₄(OR¹) where R¹ is any C₁ to C₆ branched, unbranched or cyclic alkyl group, being optionally substituted with one or more functional groups; NH₄(RCO₂), Li(RCO₂), Na(RCO₂), K(RCO₂), Rb(RCO₂), Cs(RCO₂), Mg(RCO₂)₂, Ca(RCO₂)₂, Sr(RCO₂)₂ or Ba(RCO₂)₂, where RCO₂ is selected from mesaconate, citraconate, itaconate, citrate, oxalate and methacrylate; (NH₄)₂(CO₂RCO₂), Li₂(CO₂RCO₂), Na₂(CO₂RCO₂), K₂(CO₂RCO₂), Rb₂(CO₂RCO₂), Cs₂(CO₂RCO₂), Mg(CO₂RCO₂), Ca(CO₂RCO₂), Sr(CO₂RCO₂), Ba(CO₂RCO₂), (NH₄)₂(CO₂RCO₂), where CO₂RCO₂ is selected from mesaconate, citraconate, itaconate and oxalate; (NH₄)₃(CO₂R(CO2) CO₂), Li₃(CO₂R(CO₂) CO₂), Na₃(CO₂R(CO2) CO₂), K₃(CO₂R(CO₂)CO₂), Rb₃(CO₂R(CO₂) CO₂), Cs₃(CO₂R(CO₂) CO₂), Mg₃(CO₂R(CO₂) CO₂)₂, Ca₃(CO₂R(CO₂) CO₂)₂, Sr₃(CO₂R(CO₂) CO₂)₂, Ba₃(CO₂R(CO₂)CO₂)₂, (NH₄)₃(CO₂R(CO₂)CO₂), where CO₂R(CO₂)CO₂ is selected from citrate, isocitrate and aconitate; methylamine, ethylamine, propylamine, butylamine, pentylamine, hexylamine, cyclohexylamine, aniline; and R₄NOH where R is selected from methyl, ethyl propyl, butyl. More preferably, the base is selected from one or more of the following: LiOH, NaOH, KOH, Mg(OH)₂, Ca(OH)₂, Ba(OH)₂, CsOH, Sr(OH)₂, RbOH, NH₄OH, Li₂CO₃, Na₂CO₃, K₂CO₃, Rb₂CO₃, Cs₂CO₃, MgCO₃, CaCO₃, (NH₄)₂CO₃, LiHCO₃, NaHCO₃, KHCO₃, RbHCO₃, CsHCO₃, Mg(HCO₃)₂, Ca(HCO₃)₂, Sr(HCO₃)₂, Ba(HCO₃)₂, NH₄HCO₃, Li₂O, Na₂O, K₂O, Rb₂O, Cs₂O, NH₄(RCO₂), Li(RCO₂), Na(RCO₂), K(RCO₂), Rb(RCO₂), Cs(RCO₂), Mg(RCO₂)₂, Ca(RCO₂)₂, Sr(RCO₂)₂ or Ba(RCO₂)₂, where RCO₂ is selected from itaconate, citrate, oxalate, methacrylate; (NH₄)₂(CO₂RCO₂), Li₂(CO₂RCO₂), Na₂(CO₂RCO₂), K₂(CO₂RCO₂), Rb₂(CO₂RCO₂), Cs₂(CO₂RCO₂), Mg(CO₂RCO₂), Ca(CO₂RCO₂), Sr(CO₂RCO₂), Ba(CO₂RCO₂)₁(NH₄)₂(CO₂RCO₂), where CO₂RCO₂ is selected from, mesaconate, citraconate, itaconate, oxalate; (NH₄)₃(CO₂R(CO2) CO₂), Li₃(CO₂R(CO₂) CO₂). Na₃(CO₂R(CO2) CO₂), K₃(CO₂R(CO₂) CO₂), Rb₃(CO₂R(CO₂) CO₂), Cs₃(CO₂R(CO₂) CO₂), Mg₃(CO₂R(CO₂) CO₂)₂, Ca₃(CO₂R(CO₂) CO₂)₂, where CO₂R(CO₂)CO₂ is selected from citrate, isocitrate; tetramethylammonium hydroxide and tetraethylammonium hydroxide. Most preferably, the base is selected from one or more of the following: NaOH, KOH, Ca(OH)₂, CsOH, RbOH, NH₄OH, Na₂CO₃, K₂CO₃, Rb₂CO₃, Cs₂CO₃, MgCO₃, CaCO₃, (NH₄)₂CO₃, NH₄(RCO₂), Na(RCO₂), K(RCO₂), Rb(RCO₂), Cs(RCO₂), Mg(RCO₂)₂, Ca(RCO₂)₂, Sr(RCO₂)₂ or Ba(RCO₂)₂, where RCO₂ is selected from itaconate, citrate, oxalate, methacrylate; (NH₄)₂(CO₂RCO₂), Na₂(CO₂RCO₂), K₂(CO₂RCO₂), Rb₂(CO₂RCO₂), Cs₂(CO₂RCO₂), Mg(CO₂RCO₂), Ca(CO₂RCO₂), (NH₄)₂(CO₂RCO₂), where CO₂RCO₂ is selected from mesaconate, citraconate, itaconate, oxalate; (NH₄)₃(CO₂R(CO2) CO₂), Na₃(CO₂R(CO2) CO₂), K₃(CO₂R(CO₂) CO₂), Rb₃(CO₂R(CO₂) CO₂), Cs₃(CO₂R(CO₂) CO₂), Mg₃(CO₂R(CO₂) CO₂)₂, Ca₃(CO₂R(CO₂) CO₂)₂, (NH₄)₃(CO₂R(CO₂) CO₂), where CO₂R(CO₂)CO₂ is selected from citrate, isocitrate; and tetramethylammonium hydroxide.

The catalyst may be homogeneous or heterogeneous. In one embodiment, the catalyst may be dissolved in the liquid reaction phase. However, the catalyst may be suspended on a solid support over which the reaction phase may pass. In this scenario, the reaction phase is preferably maintained in a liquid, more preferably, an aqueous phase.

Preferably, the effective mole ratio of base OH⁻:acid is between 0.001-2:1, more preferably, 0.01-1.2:1, most preferably, 0.1-1:1, especially, 0.3-1:1. By the effective mole ratio of base OH⁻ is meant the nominal molar content of OH⁻ derived from the compounds concerned.

By acid is meant the moles of acid. Thus, in the case of a monobasic base, the effective mole ratios of base OH⁻:acid will coincide with those of the compounds concerned but in the case of di or tribasic bases the effective mole ratio will not coincide with that of mole ratio of the compounds concerned.

Specifically, this may be regarded as the mole ratio of monobasic base: di or tri carboxylic acid is preferably between 0.001-2:1, more preferably, 0.01-1.2:1, most preferably, 0.1-1:1, especially, 0.3-1:1.

As the deprotonation of the acid to form the salt is only referring to a first acid deprotonation in the present invention, in the case of di or tribasic bases, the mole ratio of base above will vary accordingly.

Optionally, the methacrylic acid product whether isolated or not may be esterified to produce an ester thereof. Potential esters may be selected from C₁-C₁₂ alkyl or C₂-C₁₂ hydroxyalkyl, glycidyl, isobornyl, dimethylaminoethyl, tripropyleneglycol esters. Most preferably the alcohols or alkenes used for forming the esters may be derived from bio sources, e.g. biomethanol, bioethanol, biobutanol.

According to a second aspect of the present invention there is provided a method of preparing polymers or copolymers of methacrylic acid or methacrylic acid esters, comprising the steps of

(i) preparation of methacrylic acid or ester thereof in accordance with the first aspect of the present invention;
(ii) optional esterification of the methacrylic acid prepared in (i) to produce the methacrylic acid ester;
(iii) polymerisation of the methacrylic acid or ester thereof prepared in (i) and/or the ester prepared in (ii), optionally with one or more comonomers, to produce polymers or copolymers thereof.

Preferably, the methacrylic acid ester of (ii) above is selected from C₁-C₁₂ alkyl or C₂-C₁₂ hydroxyalkyl, glycidyl, isobornyl, dimethylaminoethyl, tripropyleneglycol esters, more preferably, ethyl, n-butyl, i-butyl, hydroxymethyl, hydroxypropyl or methyl methacrylate, most preferably, methyl methacrylate, ethyl acrylate, butyl methacrylate or butyl acrylate.

Advantageously, such polymers will have an appreciable portion if not all of the monomer residues derived from a source other than fossil fuels.

In any case, preferred comonomers include for example, monoethylenically unsaturated carboxylic acids and dicarboxylic acids and their derivatives, such as esters, amides and anhydrides.

Particularly preferred comonomers are acrylic acid, methyl acrylate, ethyl acrylate, propyl acrylate, n-butyl acrylate, iso-butyl acrylate, t-butyl acrylate, 2-ethylhexyl acrylate, hydroxyethyl acrylate, iso-bornyl acrylate, methacrylic acid, methyl methacrylate, ethyl methacrylate, propyl methacrylate, n-butyl methacrylate, iso-butyl methacrylate, t-butyl methacrylate, 2-ethylhexyl methacrylate, hydroxyethyl methacrylate, lauryl methacrylate, glycidyl methacrylate, hydroxypropyl methacrylate, iso-bornyl methacrylate, dimethylaminoethyl methacrylate, tripropyleneglycol diacrylate, styrene, α-methyl styrene, vinyl acetate, isocyanates including toluene diisocyanate and p,p′-methylene diphenyl diisocyanate, acrylonitrile, butadiene, butadiene and styrene (MBS) and ABS subject to any of the above comonomers not being the monomer selected from methacrylic acid or a methacrylic acid ester in (i) or (ii) above in any given copolymerisation of the said acid monomer or ester in (i) or a said ester monomer in (ii) with one or more of the comonomers.

It is of course also possible to use mixtures of different comonomers. The comonomers themselves may or may not be prepared by the same process as the monomers from (i) or (ii) above.

According to a further aspect of the present invention there is provided polymethacrylic acid, polymethylmethacrylate (PMMA) and polybutylmethacrylate homopolymers or copolymers formed from the method of the second aspect of the invention herein.

According to a still further aspect of the present invention there is provided a process for the production of methacrylic acid or an ester thereof comprising:—providing a source of a pre-acid selected from aconitic, citric and/or isocitric acid;

performing a decarboxylation and, if necessary, a dehydration step on the source of pre-acid by exposing the source thereof in the presence or absence of base catalyst to a sufficiently high temperature to provide itaconic, mesaconic and/or citraconic acid; and a process according to the first aspect of the present invention to provide methacrylic acid or an ester thereof.

By a source of aconitic, citric and/or isocitric acid is meant the acids and salts thereof such as group I or II metal salts thereof and includes solutions of the pre-acids and salts thereof, such as aqueous solutions thereof. Optionally, the salt may be acidified to liberate the free acid prior to, during or after the pre-acid decarboxylation step.

Preferably, the dicarboxylic acid(s) reactant(s) are exposed to the reaction conditions for a time period of at least 80 seconds.

Preferably, the dicarboxylic acid(s) reactant(s) or the source of pre-acids thereof of the present invention are exposed to the reaction conditions for a suitable time period to effect the required reaction, such as 80 seconds as defined herein but more preferably, for a time period of at least 100 seconds, yet more preferably at least about 120 seconds and most preferably at least about 150 seconds.

Typically, the dicarboxylic acid(s) reactant(s) or source of pre-acids thereof are exposed to the reaction conditions for a time period of less than about 2000 seconds, more typically less than about 1500 seconds, yet more typically less than about 1000 seconds.

Preferably, the dicarboxylic acid(s) reactant(s) or the source of pre-acids thereof of the present invention are exposed to the reaction conditions for a time period of between about 75 seconds and 3000 seconds, more preferably between about 90 seconds and 2500 seconds and most preferably between about 120 seconds and 2000 seconds.

Therefore, according to a further aspect of the present invention there is provided a process for the production of methacrylic acid by the base catalysed decarboxylation of at least one dicarboxylic acid selected from itaconic, citraconic or mesaconic acid or mixtures thereof, wherein the decarboxylation is carried out in the temperature range between 200 and 239° C. and the dicarboxylic acid(s) reactant(s) are exposed to the reaction conditions for a time period of at least 80 seconds.

Advantageously, in this temperature range high selectivities can be achieved at residence times sufficient to allow heating of the reactants in the reaction medium.

Preferably, the dicarboxylic acid(s) reactant(s) or the source of pre-acids thereof of the present invention are dissolved in water so that the reaction occurs under aqueous conditions.

It will be clear from the way in which the above reactions are defined that if the source of pre-acid is decarboxylated and, if necessary, dehydrated in a reaction medium then the reaction medium may simultaneously be effecting base catalysed decarboxylation of the at least one dicarboxylic acid selected from itaconic, citraconic or mesaconic acid or mixtures thereof produced from the source of pre-acid according to the first aspect of the invention. Accordingly, the decarboxylation and if necessary, dehydration of the source of pre-acid and the base catalysed decarboxylation of the at least one dicarboxylic acid may take place in one reaction medium i.e. the two processes may take place as a so called “one pot” process. However, it is preferred that the source of pre-acid is decarboxylated and, if necessary, dehydrated substantially without base catalysis so that the decarboxylation and if necessary, dehydration of the source of pre-acid and the base catalysed decarboxylation of the at least one dicarboxylic acid take place in separate steps.

Preferably, the concentration of the dicarboxylic acid reactant(s) is at least 0.1M, preferably in an aqueous source thereof; more preferably at least about 0.2M, preferably in an aqueous source thereof; most preferably at least about 0.3M, preferably in an aqueous source thereof, especially, at least about 0.5M. Generally, the aqueous source is an aqueous solution.

Preferably, the concentration of the dicarboxylic acid reactant(s) is less than about 10M, more preferably, less than 8M, preferably in an aqueous source thereof; more preferably, less than about 5M, preferably in an aqueous source thereof; more preferably less than about 3M, preferably in an aqueous source thereof.

Preferably, the concentration of the dicarboxylic acid reactant(s) is in the range 0.05M-20, typically, 0.05-10M, more preferably, 0.1M-5M, most preferably, 0.3M-3M.

The base catalyst may be dissolvable in a liquid medium, which may be water or the base catalyst may be heterogeneous. The base catalyst may be dissolvable in the reaction mixture so that reaction is effected by exposing the reactants to the temperatures given herein which are temperatures in excess of that at which base catalysed decarboxylation of the reactant(s) to methacrylic acid and/or the source of pre-acids to the dicarboxylic acids will occur. The catalyst may be in an aqueous solution. Accordingly, the catalyst may be homogenous or heterogeneous but is typically homogenous. Preferably, the concentration of the catalyst in the reaction mixture (including the decomposition of the source of pre-acid mixture) is at least 0.1M or greater, preferably in an aqueous source thereof; more preferably at least about 0.2M, preferably in an aqueous source thereof; more preferably at least about 0.3M.

Preferably, the concentration of the catalyst in the reaction mixture (including the decomposition of the source of pre-acid mixture) is less than about 10M, more preferably, less than about 5M, more preferably less than about 2M and, in any case, preferably less than or equal to that which would amount to a saturated solution at the temperature and pressure of the reaction.

Preferably, the mole concentration of OH⁻ in the aqueous reaction medium or optionally source of pre-acid decomposition is in the range 0.05M-20M, more preferably, 0.1-5M, most preferably, 0.2M-3M.

Preferably, the reaction conditions are weakly acidic. Preferably, the reaction pH is between about 2 and 9, more preferably between about 3 and about 6.

For the avoidance of doubt, by the term itaconic acid, it is meant the following compound of formula (i)

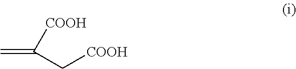

For the avoidance of doubt, by the term citraconic acid, it is meant the following compound of formula (ii)

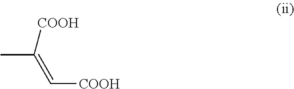

For the avoidance of doubt, by the term mesaconic acid, it is meant the following compound of formula (iii)

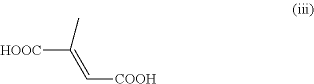

As mentioned above, the process of the present invention may be homogenous or heterogeneous. In addition, the process may be a batch or continuous process.

Advantageously, one by-product in the production of MAA may be hydroxy isobutyric acid (HIB) which exists in equilibrium with the product MAA at the conditions used for decomposition of the dicarboxylic acids. Accordingly, partial or total separation of the MAA from the products of the decomposition reaction shifts the equilibrium from HIB to MAA thus generating further MAA during the process or in subsequent processing of the solution after separation of MAA.

As mentioned above, the source of pre-acid such as citric acid, isocitric acid or aconitic acid preferably decomposes under suitable conditions of temperature and pressure and optionally in the presence of base catalyst to one of the dicarboxylic acids of the invention. Suitable conditions for this decomposition are less than 350° C., typically, less than 330° C., more preferably, at up to 310° C., most preferably at up to 300° C. In any case, a preferred lower temperature for the decomposition is 100° C. Preferred temperature ranges for the source of pre-acid decomposition are between 110 and up to 349° C., more preferably, between 120 and 300° C., most preferably, between 130 and 280° C., especially between 140 and 260° C.

Preferably, the source of pre-acid decomposition reaction takes place at a temperature at which the aqueous reaction medium is in the liquid phase.

To maintain the reactants in the liquid phase under the above source of pre-acid decomposition temperature conditions the decarboxylation reaction is carried out at suitable pressures at or in excess of atmospheric pressure. Suitable pressures which will maintain the reactants in the liquid phase in the above temperature ranges are greater than 15 psia, more suitably, greater than 20 psia, most suitably, greater than 25 psia and in any case at a higher pressure than that below which the reactant medium will boil. There is no upper limit of pressure but the skilled person will operate within practical limits and within apparatus tolerances, for instance, at less than 10,000 psia, more typically, at less than 5,000 psia, most typically, at less than 4000 psia.

Preferably, the source of pre-acid decomposition reaction is at a pressure of between about 15 and 10000 psia. More preferably, the reaction is at a pressure of between about 20 and 5000 psia and yet more preferably between about 25 and 3000 psia.

In a preferred embodiment, the source of pre-acid decomposition reaction is at a pressure at which the reaction medium is in the liquid phase.

Preferably, the source of pre-acid decomposition reaction is at a temperature and pressure at which the aqueous reaction medium is in the liquid phase.

All of the features contained herein may be combined with any of the above aspects, in any combination.

For a better understanding of the invention, and to show how embodiments of the same may be carried into effect, reference will now be made, by way of example, to the following examples.

### EXAMPLES

A series of experiments were conducted investigating the decomposition of itaconic, citraconic and mesaconic acids to form methacrylic acid at various temperatures and residence times.

The chemicals used in these experiments were all obtained from Sigma Aldrich; Itaconic acid (>=99%) (Catalogue number: I2,920-4); citraconic acid (98+%) (Catalogue number C82604); mesaconic acid (99%) (Catalogue number: 13,104-0) and sodium hydroxide (>98%) (Catalogue number S5881).

The procedure for these experiments is as follows. The feed solution for the experiment was prepared by mixing together a di-carboxylic acid (either itaconic, citraconic or mesaconic acid) (65 g, 0.5 moles) and sodium hydroxide (20 g, 0.5 moles). The two solids were then dissolved in 915 g de-ionised water to give a total feed solution weight of 1 kg.

The reaction solution was then fed into the ThalesNano X-Cube Flash apparatus at the required flow rate to obtain 120, 240, 366, 480, 600 and 870 seconds residence times. Every experiment was carried out at a set pressure of 150 bar (2176 psi). The temperature of the reactor was adjusted according to the requirements of each experiment.

### X-Cube Flash Operation

Ensure both pump lines are attached and immersed in solvent. Set the reaction pressure to the required pressure (150 bar). Set the reaction temperature to the required temperature. Ensure that the feed line for pump 1 is inserted into the reactant feed solution bottle. Select pump 1 and set to the required flow rate of the feed solution to achieve the desired residence time of the solution in the reactor. Start the experiment and run the pump 1 for 20 minutes. After running the pump for 20 minutes start to collect the liquid sample exiting the X-cube. After sufficient reactor exit has been collected, the X-Cube will need to be flushed with water to avoid cross contamination between experimental samples. Ensure that the feed line for pump 2 is inserted into the water feed bottle. Switch the liquid feed to the reactor from that fed from pump 1 (reactant solution) to that fed from pump (water). Run the pump for 20 minutes so that no reactant solution is left in the reactor.

### Analysis

All reaction exit solutions were analysed by ¹H NMR spectroscopy. All samples were run on either a 500 MhZ JOEL spectrometer or a 300 Mhz JOEL spectrometer. All NMR spectra that were observed were analysed and the relative mol % of the individual components calculated on the basis of the observed integrals. A series of decarboxylation experiments were carried out on itaconic (IC), citraconic (CC) and mesaconic (MC) acid at various temperatures and residence times according to the above procedure. The results are shown below.

TABLE 1
Conversion and Selectivity at Various Temperatures and
Residence Times for Citraconic acid Decarboxylation

| | | | relative | | | | | | | | |
|---|---|---|---|---|---|---|---|---|---|---|---|
| | Residence | | mol % | | | | | | | | mol % |
| Ex | time/secs | Temp ° C. | MC | IC | CC | PC | MAA | HIB | TBP | % Conv. | Sel. |
| 1 | 600.00 | 200.00 | 13.94 | 12.33 | 34.20 | 8.90 | 26.86 | 3.19 | 0.57 | 30.05 | 97.91% |
| 2 | 480.00 | 200.00 | 9.35 | 18.33 | 50.51 | 6.49 | 14.17 | 1.05 | 0.10 | 15.22 | 99.29% |
| 3 | 366.00 | 200.00 | 1.28 | 20.60 | 71.15 | 3.89 | 3.07 | 0.01 | 0.00 | 3.08 | 100.00% |
| 4 | 366.00 | 210.00 | 4.05 | 19.42 | 60.43 | 7.57 | 8.14 | 0.39 | 0.00 | 8.53 | 100.00% |
| 5 | 366.00 | 220.00 | 11.21 | 15.47 | 43.68 | 8.27 | 19.55 | 1.31 | 0.51 | 20.86 | 97.47% |
| 6 | 366.00 | 230.00 | 12.99 | 12.56 | 34.84 | 2.13 | 32.78 | 3.88 | 0.83 | 36.65 | 97.54% |
| 7 | 240.00 | 210.00 | 0.38 | 18.43 | 77.02 | 1.98 | 1.97 | 0.22 | 0.00 | 2.19 | 100.00% |
| 8 | 240.00 | 220.00 | 2.68 | 21.01 | 69.14 | 3.72 | 3.13 | 0.31 | 0.01 | 3.43 | 99.65% |
| 9 | 240.00 | 230.00 | 5.11 | 17.48 | 58.94 | 9.73 | 8.06 | 0.58 | 0.09 | 8.64 | 98.84% |
| 10 | 120.00 | 220.00 | 0.00 | 18.09 | 81.09 | 0.00 | 0.82 | 0.00 | 0.00 | 0.82 | 100.00% |
| 11 | 120.00 | 230.00 | 2.58 | 22.88 | 66.48 | 5.06 | 2.99 | 0.00 | 0.00 | 2.99 | 100.00% |

Key: MC Mesaconic Acid
IC Itaconic Acid
CC Citraconic Acid
PC Paraconic Acid
MAA Methacrylic acid
HIB Hydroxyisobutyric acid
TBP Total Other By-Products
Conv. Conversion
Sel. Selectivity

TABLE 2
Conversion and Selectivity at Various Temperatures and
Residence times for Itaconic acid Decarboxylation

| | | | relative | | | | | | | | |
|---|---|---|---|---|---|---|---|---|---|---|---|
| | Residence | | mol % | | | | | | | | mol % |
| Ex | time/secs | Temp ° C. | MC | IC | CC | PC | MAA | HIB | TBP | % Conv. | Sel. |
| 12 | 870.00 | 200.00 | 10.55 | 6.79 | 17.92 | 2.26 | 47.26 | 10.59 | 4.64 | 57.85 | 91.07% |
| 13 | 870.00 | 210.00 | 0.65 | 0.00 | 3.81 | 0.00 | 66.75 | 21.81 | 6.97 | 88.56 | 90.54% |
| 14 | 480.00 | 200° C. | 12.98 | 15.87 | 38.99 | 10.63 | 21.06 | 0.00 | 0.46 | 21.06 | 97.84% |
| 15 | 480.00 | 210° C. | 12.63 | 9.79 | 32.41 | 6.89 | 36.99 | 0.00 | 1.29 | 36.99 | 96.62% |
| 16 | 480.00 | 220.00 | 11.32 | 4.76 | 19.47 | 5.45 | 52.86 | 2.23 | 3.92 | 55.09 | 93.10% |
| 17 | 366.00 | 200° C. | 7.79 | 20.72 | 46.64 | 13.30 | 11.56 | 0.00 | 0.00 | 11.56 | 100.00% |
| 18 | 366.00 | 210° C. | 11.89 | 16.66 | 43.86 | 11.36 | 16.24 | 0.00 | 0.00 | 16.24 | 100.00% |
| 19 | 366.00 | 220° C. | 14.91 | 13.66 | 35.71 | 9.14 | 25.77 | 0.00 | 0.81 | 25.77 | 96.96% |
| 20 | 366.00 | 230.00 | 15.50 | 11.25 | 27.84 | 1.56 | 41.20 | 0.29 | 2.35 | 41.49 | 94.60% |
| 21 | 240.00 | 200.00 | 4.41 | 47.43 | 35.99 | 7.62 | 4.55 | 0.00 | 0.00 | 4.55 | 100.00% |
| 22 | 240.00 | 210.00 | 7.11 | 29.43 | 45.08 | 10.88 | 7.50 | 0.00 | 0.00 | 7.50 | 100.00% |
| 23 | 240.00 | 220.00 | 8.34 | 20.47 | 47.02 | 13.34 | 10.83 | 0.00 | 0.00 | 10.83 | 100.00% |
| 24 | 240.00 | 230.00 | 10.57 | 18.55 | 43.46 | 9.54 | 17.64 | 0.00 | 0.24 | 17.64 | 98.66% |
| 25 | 120.00 | 200.00 | 2.50 | 60.78 | 27.64 | 7.19 | 1.88 | 0.00 | 0.00 | 1.88 | 100.00% |
| 26 | 120.00 | 210.00 | 3.74 | 40.63 | 39.82 | 12.02 | 3.79 | 0.00 | 0.00 | 3.79 | 100.00% |
| 27 | 120.00 | 220.00 | 6.39 | 26.17 | 50.36 | 9.74 | 7.33 | 0.00 | 0.00 | 7.33 | 100.00% |
| 28 | 120.00 | 230.00 | 9.85 | 18.56 | 49.61 | 11.50 | 10.49 | 0.00 | 0.00 | 10.49 | 100.00% |

TABLE 3
Conversion and Selectivity at Various Temperatures and
Residence times for Mesaconic acid Decarboxylation

| | | | relative | | | | | | | | |
|---|---|---|---|---|---|---|---|---|---|---|---|
| | Residence | | mol % | | | | | | | | mol % |
| Ex | time/secs | Temp ° C. | MC | IC | CC | PC | MAA | HIB | TBP | % Conv. | Sel. |
| 29 | 600.00 | 200.00 | 13.55 | 6.55 | 18.65 | 1.70 | 48.36 | 7.26 | 3.93 | 55.62 | 92.48% |
| 30 | 600.00 | 210.00 | 13.44 | 6.64 | 20.99 | 0.00 | 47.21 | 8.97 | 2.75 | 56.18 | 94.50% |
| 31 | 600.00 | 220.00 | 6.91 | 3.76 | 11.52 | 0.00 | 59.28 | 14.22 | 4.31 | 73.50 | 93.22% |
| 32 | 480.00 | 200.00 | 50.09 | 11.46 | 23.63 | 5.62 | 8.42 | 0.78 | 0.00 | 9.20 | 100.00% |
| 33 | 480.00 | 210.00 | 29.03 | 12.70 | 30.82 | 3.20 | 21.04 | 3.21 | 0.00 | 24.25 | 100.00% |
| 34 | 480.00 | 220.00 | 17.03 | 9.29 | 23.40 | 5.88 | 36.31 | 7.15 | 0.94 | 43.46 | 97.47% |
| 35 | 480.00 | 230.00 | 9.91 | 3.98 | 13.72 | 0.00 | 55.95 | 11.93 | 4.50 | 67.89 | 92.56% |
| 36 | 366.00 | 200.00 | 81.09 | 8.00 | 8.73 | 0.30 | 1.87 | 0.00 | 0.00 | 1.87 | 100.00% |
| 37 | 366.00 | 210.00 | 64.91 | 10.50 | 18.17 | 2.64 | 3.78 | 0.00 | 0.00 | 3.78 | 100.00% |
| 38 | 366.00 | 220.00 | 49.20 | 13.10 | 26.28 | 0.81 | 10.62 | 0.00 | 0.00 | 10.62 | 100.00% |
| 39 | 366.00 | 230.00 | 26.86 | 12.28 | 29.79 | 4.33 | 24.40 | 2.11 | 0.22 | 26.51 | 99.11% |
| 40 | 240.00 | 210.00 | 88.02 | 6.28 | 5.62 | 0.00 | 0.08 | 0.00 | 0.00 | 0.08 | 100.00% |
| 41 | 240.00 | 220.00 | 76.17 | 9.10 | 12.94 | 0.00 | 1.79 | 0.00 | 0.00 | 1.79 | 100.00% |
| 42 | 240.00 | 230.00 | 58.27 | 10.00 | 19.04 | 8.48 | 4.22 | 0.00 | 0.00 | 4.22 | 100.00% |
| 43 | 120.00 | 230.00 | 86.96 | 6.57 | 5.84 | 0.00 | 0.63 | 0.00 | 0.00 | 0.63 | 100.00% |

### Example 44

### Decarboxylation of Itaconic Acid Followed by Purification

A decarboxylation reaction solution was made by mixing together itaconic acid, sodium hydroxide and water. This was then fed through the X-Cube flash at 220° C. and 600 seconds residence time at a pressure of 150 bar. The relative amounts of the three components are shown below

Relative Composition

| | |
|---|---|
| IC | 98 g |
| NaOH | 30 g |
| Water | 872 g |

In total 1186 g of the feed composition material was fed into the reactor this gave 1046 g of reactor exit.

Reaction composition, all in relative mol %

| Residence | | | | | | | | |
|---|---|---|---|---|---|---|---|---|
| time/sec | T/° C. | MC | IC | CC | PC | MAA | HIB | TBP |
| 600.00 | 220 | 3.71 | 1.25 | 4.61 | 1.95 | 64.22 | 14.83 | 9.44 |

The reactor exit solution was then placed into a 1 L flask and then heated under vacuum until it had decreased in volume from 1 L to 500 ml.

The mixture was then mixed with Itaconic acid (98 g) and stirred for 1 hour. Then the resultant mixture was then distilled under air to a final oil temperature of 180° C. during this time a colourless liquid with a boiling point range of 100-104° C. was collected. The solid residue after distillation was orange/brown in colour. The total weight of the distillate was 257 g. A small sample was taken for GC analysis.

The remaining aqueous distillate was placed into a 1 L flask (weight added=245.7 g) and toluene was added (246.6 g). The resultant bi-phasic mixture was then shaken rapidly for five minutes and then left to stand overnight. The two phases were then separated.

The organic phase was then analysed by GC, this indicated that the organic liquid was 4.0% MAA and 96% Toluene. Based upon the weight % MAA in the organic extract 10.6 g of MAA was transferred into the organic phase. Purification is completed by removal of the toluene employing reduced pressure distillation.

### Example 45

### Decarboxylation of Mesaconic Acid Followed by Purification

The solution was made by mixing together mesaconic acid, sodium hydroxide and water. The solution was then fed through the X-Cube flash at 220° C. and 600 seconds residence time at a pressure of 150 bar. The relative amounts of the three components is shown below

| | |
|---|---|
| MC | 98 g |
| NaOH | 30 g |
| Water | 872 g |

In total 1150 g of the feed composition material was fed into the reactor this gave 1024 g of reactor exit.

Reaction composition, all in relative mol %

| Residence | | | | | | | | |
|---|---|---|---|---|---|---|---|---|
| time/sec | T/° C. | MC | IC | CC | PCA | MAA | HIB | TBP |
| 600.00 | 210 | 13.44 | 6.64 | 20.99 | 0.00 | 47.21 | 8.97 | 2.75 |

The reactor exit solution was then placed into a 1 L flask and then heated under vacuum until it had decreased in volume from 1 L to 500 ml.

The mixture was then mixed with further mesaconic acid (98 g) and stirred for 1 hour. Then the resultant mixture was then distilled under air to a final oil temperature of 180° C. during this time a colourless liquid with a boiling point range of 100-104° C. was collected. The solid residue after distillation was orange/brown in colour. The total weight of the distillate was 512 g. A small sample was taken for GC analysis.

The remaining aqueous distillate was placed into a 1 L flask (weight added=497 g) and toluene was added (500 g). The resultant bi-phasic mixture was then shaken rapidly for five minutes and then left to stand overnight. The two phases were then separated.

The organic phase was then analysed by GC, this indicated that the organic liquid was 3.1% MAA and 96.9% Toluene. Based upon the weight % MAA in the organic extract 16.1 g of MAA was transferred into the organic phase. Purification is completed by removal of the toluene employing reduced pressure distillation.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A process for the production of methacrylic acid or esters thereof by the base catalysed decarboxylation of at least one dicarboxylic acid selected from itaconic, citraconic or mesaconic acid or mixtures thereof in an aqueous reaction medium, wherein the decarboxylation is carried out at a temperature in the range from 200° C. and up to 239° C. and wherein the methacrylic acid is isolated from the aqueous reaction medium by a purification process which does not include introducing an organic solvent to the said aqueous reaction medium for solvent extraction of the methacrylic acid into an organic phase.

2. A method or process according to any claim 1, wherein the dicarboxylic acid(s) reactant(s) or the source of pre-acids thereof are exposed to the reaction conditions for a time period of between about 75 seconds and 3000 seconds.

3. A process for the production of methacrylic acid by the base catalysed decarboxylation of at least one dicarboxylic acid selected from itaconic, citraconic or mesaconic acid or mixtures thereof, wherein the decarboxylation is carried out in the temperature range between 200 and 239° C. and the dicarboxylic acid(s) reactant(s) are exposed to the reaction conditions for a time period of at least 80 seconds.

4. A process according to claim 1, wherein the decarboxylation reaction is carried out at a pressure of between about 225 and 10000 psia.

5. A process according to claim 1, wherein suitable processes to isolate the methacrylic acid from the aqueous reaction medium are selected from distillation and fractional crystallisation wherein crystallisation can include crystallisation of the free acid or crystallisation of a salt of the acid such as the group I and II metal salt, for example the calcium salt, followed by acidification to regenerate the free MAA, wherein crystallisation may be preceded by suitable separation such as ion exchange chromatography, wherein crystallisation may include bipolar membrane electrodialysis (BPMED) to increase the purity of the MAA prior to crystallisation, for example by forming MAA and NaOH from sodium methacrylate, and wherein the isolation technique may involve esterification to the alkyl ester followed by distillation and optional subsequent hydrolysis to regenerate the MAA.

6. A process according to claim 1, wherein the catalyst comprises a source of OH⁻ ions.

7. A process according to claim 1, wherein the base catalyst is selected from the group consisting of a metal oxide, hydroxide, carbonate, acetate (ethanoate), alkoxide, hydrogencarbonate; or salt of a decomposable di- or tri-carboxylic acid; or a quaternary ammonium compound of one of the above; or one or more amines; more preferably a Group I or Group II metal oxide, hydroxide, carbonate, acetate, alkoxide, hydrogencarbonate or salt of a di- or tri-carboxylic acid or methacrylic acid.

8. A process according to claim 7, wherein the base catalyst is selected from one or more of the group consisting of LiOH, NaOH, KOH, Mg(OH)₂, Ca(OH)₂, Ba(OH)₂, CsOH, Sr(OH)₂, RbOH, NH₄OH, Li₂CO₃, Na₂CO₃, K₂CO₃, Rb₂CO₃, Cs₂CO₃, MgCO₃, CaCO₃, SrCO₃, BaCO₃, (NH₄)₂CO₃, LiHCO₃, NaHCO₃, KHCO₃, RbHCO₃, CsHCO₃, Mg(HCO₃)₂, Ca(HCO₃)₂, Sr(HCO₃)₂, Ba(HCO₃)₂, NH₄HCO₃, Li₂O, Na₂O, K₂O, Rb₂O, Cs₂O, MgO, CaO, SrO, BaO, Li(OR¹), Na(OR¹), K(OR¹), Rb(OR¹), Cs(OR¹), Mg(OR¹)₂, Ca(OR¹)₂, Sr(OR¹)₂, Ba(OR¹)₂, NH₄(OR¹) where R¹ is any C₁ to C₆ branched, unbranched or cyclic alkyl group, being optionally substituted with one or more functional groups; NH₄(RCO₂), Li(RCO₂), Na(RCO₂), K(RCO₂), Rb(RCO₂), Cs(RCO₂), Mg(RCO₂)₂, Ca(RCO₂)₂, Sr(RCO₂)₂ or Ba(RCO₂)₂, where RCO₂ is selected from mesaconate, citraconate, itaconate, citrate, oxalate and methacrylate; (NH₄)₂(CO₂RCO₂), Li₂(CO₂RCO₂), Na₂(CO₂RCO₂), K₂(CO₂RCO₂), Rb₂(CO₂RCO₂), Cs₂(CO₂RCO₂), Mg(CO₂RCO₂), Ca(CO₂RCO₂), Sr(CO₂RCO₂), Ba(CO₂RCO₂), (NH₄)₂(CO₂RCO₂), where CO₂RCO₂ is selected from mesaconate, citraconate, itaconate and oxalate; (NH₄)₃(CO₂R(CO₂)CO₂), Li₃(CO₂R(CO₂)CO₂), Na₃(CO₂R(CO₂)CO₂), K₃(CO₂R(CO₂)CO₂), Rb₃(CO₂R(CO₂)CO₂), Cs₃(CO₂R(CO₂)CO₂), Mg₃(CO₂R(CO₂)CO₂)₂, Ca₃(CO₂R(CO₂)CO₂)₂, Sr₃(CO₂R(CO₂)CO₂)₂, Ba₃(CO₂R(CO₂)CO₂)₂, (NH₄)₃(CO₂R(CO₂)CO₂), where CO₂R(CO₂)CO₂ is selected from citrate, isocitrate and aconitate; methylamine, ethylamine, propylamine, butylamine, pentylamine, hexylamine, cyclohexylamine, aniline; and R₄NOH where R is selected from methyl, ethyl propyl, butyl. More preferably, the base is selected from one or more of the following: LiOH, NaOH, KOH, Mg(OH)₂, Ca(OH)₂, Ba(OH)₂, CsOH, Sr(OH)₂, RbOH, NH₄OH, Li₂CO₃, Na₂CO₃, K₂CO₃, Rb₂CO₃, Cs₂CO₃, MgCO₃, CaCO₃, (NH₄)₂CO₃, LiHCO₃, NaHCO₃, KHCO₃, RbHCO₃, CsHCO₃, Mg(HCO₃)₂, Ca(HCO₃)₂, Sr(HCO₃)₂, Ba(HCO₃)₂, NH₄HCO₃, Li₂O, Na₂O, K₂O, Rb₂O, Cs₂O, NH₄(RCO₂), Li(RCO₂), Na(RCO₂), K(RCO₂), Rb(RCO₂), Cs(RCO₂), Mg(RCO₂)₂, Ca(RCO₂)₂, Sr(RCO₂)₂ or Ba(RCO₂)₂, where RCO₂ is selected from itaconate, citrate, oxalate, methacrylate; (NH₄)₂(CO₂RCO₂), Li₂(CO₂RCO₂), Na₂(CO₂RCO₂), K₂(CO₂RCO₂), Rb₂(CO₂RCO₂), Cs₂(CO₂RCO₂), Mg(CO₂RCO₂), Ca(CO₂RCO₂), Sr(CO₂RCO₂), Ba(CO₂RCO₂), (NH₄)₂(CO₂RCO₂), where CO₂RCO₂ is selected from, mesaconate, citraconate, itaconate, oxalate; (NH₄)₃(CO₂R(CO₂)CO₂), Li₃(CO₂R(CO₂)CO₂), Na₃(CO₂R(CO₂)CO₂), K₃(CO₂R(CO₂)CO₂), Rb₃(CO₂R(CO₂)CO₂), Cs₃(CO₂R(CO₂)CO₂), Mg₃(CO₂R(CO₂)CO₂)₂, Ca₃(CO₂R(CO₂)CO₂)₂, Sr₃(CO₂R(CO₂)CO₂)₂, Ba₃(CO₂R(CO₂)CO₂)₂, (NH₄)₃(CO₂R(CO₂)CO₂), where CO₂R(CO₂)CO₂ is selected from citrate, isocitrate; tetramethylammonium hydroxide and tetraethylammonium hydroxide. Most preferably, the base is selected from one or more of the following: NaOH, KOH, Ca(OH)₂, CsOH, RbOH, NH₄OH, Na₂CO₃, K₂CO₃, Rb₂CO₃, Cs₂CO₃, MgCO₃, CaCO₃, (NH₄)₂CO₃, NH₄(RCO₂), Na(RCO₂), K(RCO₂), Rb(RCO₂), Cs(RCO₂), Mg(RCO₂)₂, Ca(RCO₂)₂, Sr(RCO₂)₂ or Ba(RCO₂)₂, where RCO₂ is selected from itaconate, citrate, oxalate, methacrylate; (NH₄)₂(CO₂RCO₂), Na₂(CO₂RCO₂), K₂(CO₂RCO₂), Rb₂(CO₂RCO₂), Cs₂(CO₂RCO₂), Mg(CO₂RCO₂), Ca(CO₂RCO₂), (NH₄)₂(CO₂RCO₂), where CO₂RCO₂ is selected from mesaconate, citraconate, itaconate, oxalate; (NH₄)₃(CO₂R(CO₂)CO₂), Na₃(CO₂R(CO₂)CO₂), K₃(CO₂R(CO₂)CO₂), Rb₃(CO₂R(CO₂)CO₂), Cs₃(CO₂R(CO₂)CO₂), Mg₃(CO₂R(CO₂)CO₂)₂, Ca₃(CO₂R(CO₂)CO₂)₂, (NH₄)₃(CO₂R(CO₂)CO₂), where CO₂R(CO₂)CO₂ is selected from citrate, isocitrate; and tetramethylammonium hydroxide.

9. A process according to claim 1, wherein the effective mole ratio of base OH⁻:acid is between 0.001-2:1.

10. A process according to claim 1, wherein the concentration of the dicarboxylic acid reactant(s) is in the range 0.05M-20M.

11. A process according to claim 1, wherein the concentration of the catalyst in the reaction mixture (including the decomposition of the source of pre-acid mixture) is at least 0.1M.

12. A process according to claim 1, wherein the concentration of the catalyst in the reaction mixture (including the decomposition of the source of pre-acid mixture) is less than about 10M.

13. A process according to claim 1, wherein the reaction pH is between about 2 and 9.

14. A process for the production of methacrylic acid or an ester thereof comprising:—providing a source of a pre-acid selected from aconitic, citric and/or isocitric acid; performing a decarboxylation and, if necessary, a dehydration step on the source of pre-acid by exposing the source thereof in the presence or absence of base catalyst to a sufficiently high temperature to provide itaconic, mesaconic and/or citraconic acid; and a process according to claim 1 to provide methacrylic acid or an ester thereof.

15. A process according to claim 14, wherein the temperature ranges for the source of pre-acid decomposition are between 110 and up to 349° C.

16. A process according to claim 14, wherein the pre-acid decomposition reaction is at a pressure of between about 15 and 10000 psia.

17. A method of preparing polymers or copolymers of methacrylic acid or methacrylic acid esters, comprising the steps of
(i) preparation of methacrylic acid or ester thereof in accordance with the process of claim 1;
(ii) optional esterification of the methacrylic acid prepared in (i) to produce the methacrylic acid ester;
(iii) polymerisation of the methacrylic acid or ester thereof prepared in (i) and/or the ester prepared in (ii), optionally with one or more comonomers, to produce polymers or copolymers thereof.

18. A method according to claim 17, wherein the methacrylic acid ester of (ii) above is selected from C₁-C₁₂ alkyl or C₂-C₁₂ hydroxyalkyl, glycidyl, isobornyl, dimethylaminoethyl, tripropyleneglycol esters.

19. A method according to claim 17, wherein the comonomers are selected from the group consisting of monoethylenically unsaturated carboxylic acids, dicarboxylic acids and their derivatives, such as esters, amides and anhydrides.

20. A method according to claim 19, wherein the comonomers are selected from the group consisting of acrylic acid, methyl acrylate, ethyl acrylate, propyl acrylate, n-butyl acrylate, iso-butyl acrylate, t-butyl acrylate, 2-ethylhexyl acrylate, hydroxyethyl acrylate, iso-bornyl acrylate, methacrylic acid, methyl methacrylate, ethyl methacrylate, propyl methacrylate, n-butyl methacrylate, iso-butyl methacrylate, t-butyl methacrylate, 2-ethylhexyl methacrylate, hydroxyethyl methacrylate, lauryl methacrylate, glycidyl methacrylate, hydroxypropyl methacrylate, iso-bornyl methacrylate, dimethylaminoethyl methacrylate, tripropyleneglycol diacrylate, styrene, α-methyl styrene, vinyl acetate, isocyanates including toluene diisocyanate and p,p′-methylene diphenyl diisocyanate, acrylonitrile, butadiene, butadiene and styrene (MBS) and ABS subject to any of the above comonomers not being the monomer selected from methacrylic acid or a methacrylic acid ester in (i) or (ii) above in any given copolymerisation of the said acid monomer or ester in (i) or a said ester monomer in (ii) with one or more of the comonomers.

21. A method according to claim 17, wherein the itaconic, citraconic or mesaconic acids are produced from a source of pre-acids.

22. (canceled)

23. (canceled)
